(19) United States
(12) Patent Application Publication
(10) Pub. No.: US 2013/0209551 A1
(43) Pub. Date: Aug. 15, 2013

(54) PENTAPEPTIDE DERIVATIVES FOR PROMOTING HAIR GROWTH

(71) Applicant: Markus Lüthy, Arlesheim (CH)

(72) Inventor: Markus Lüthy, Arlesheim (CH)

(21) Appl. No.: 13/825,831

(22) PCT Filed: Sep. 30, 2011
(86) PCT No.: PCT/EP2011/067093

§ 371 Date: Mar. 25, 2013

(30) Foreign Application Priority Data
Oct. 1, 2010 (CH) 01609/10

(51) Int. Cl. C07K 7/06; A61K 45/06; A61K 38/08

(52) U.S. Cl. CPC C07K 7/06; A61K 38/08; A61K 45/06
USPC 424/450; 530/330; 514/20.7; 424/490

(57) ABSTRACT

The invention relates to substituted pentapeptide compounds of the formula R¹-Gly-Pro-Ile-Gly-A₁ (I), where A₁ is Ser or Thr and R¹ denotes —C(═O)—H, —C(═O)—C₁-C₂₄ alkyl, —C(═O)—CH₂CH(OH)CH₂N⁺(CH₃)₃ (carnitinoyl), 1,2-Dithiolane-3-pentanoyl (liponoyl), —C(═O)-(CH₂)₂NHC(═O)CH(OH)C(CH₃)₂CH₂OH (pantothenoyl) or (II) (biotinyl) in free form or in salt form, to the use thereof to produce cosmetic dermatologically active compositions, to said compositions, to the use thereof as hair growth promoters, and to a cosmetic method for promoting hair growth.

The invention relates to substituted pentapeptide compounds, the use thereof to produce cosmetic dermatologically active compositions, said compositions, the use thereof as hair growth promoters, and to a cosmetic method for promoting hair growth.

Humans have approximately 100,000 to 150,000 hairs. There are three distinguishable phases in the hair-growth cycle that every individual hair goes through—namely, the anagen, catagen and telogen phases. The anagen phase (active phase or growth phase) lasting several years, during which hairs become longer, is followed by a very brief and transitory catagen phase lasting a few weeks. The follicle atrophies during this phase, and the incorporation of the follicle in the dermis appears increasingly pushed upward. Finally, the telogen phase lasting several months is the resting phase of the follicle during which the hair is shedded. A new follicle is generated at the end of this resting phase in the same location, and the cycle begins anew.

In a healthy human person, this cycle repeats at intervals of 3 to 6 years with the result that between 60 and 100 hairs are shedded per day. On the other hand, in people with a disrupted cycle, the proportion of hairs in the anagen phase decreases, while the proportion of hairs in the catagen and telogen phases increases, such that the number of shedded hair increases abnormally.

Hair loss can cause serious psychological problems in sufferers, including a loss of self-confidence. Therefore, it is crucial to treat excessive hair loss from the very beginning.

Various approaches have been tried throughout the world to treat hair loss and promote hair growth in men and women. The majority of the active substances against hair loss known to date, as well as nutrients for hair have failed to demonstrate sufficiently potent effects; moreover, they frequency suffer from the risk of toxic side effects for the human body.

Minoxidil, which is the widest known hair growth promoter in the United States that is approved by the FDA, is intended for external use and induces the anagen phase to restart from the telogen phase, maintaining the same continuously constant (British Journal of Dermatology, 150, 186-194, 2004). However, minoxidil only demonstrates weak efficacy, and specifically only provided it is applied without interruption twice daily and left on the scalp for three to four hours each time. The inadequate efficacy is accompanied by side effects, such as itching or skin irritation.

Orally applicable active substances, like finasteride, that inhibit the 5-alpha reductase are marketed as FDA-approved hair growth promoters. However, they are expensive and show side effects such as impotence or teratogeneity.

Natural active substances are, inter alia, the ingredients of the Sophora root that increase the hair growth factors IGF-1 and KGF; procyanidins that increase the thickness and density of hair; vitamin A that stimulates the formation of new hair cells; vitamin D that stimulates hair growth; arginine for promoting hair cell growth; creatine ethyl ester that provides energy for hair cells; and L-carnitine promoting improved nutrient transport. However, all of these substances lack sufficient efficacy for treating alopecia.

Among peptidic substances that have a positive influence on hair growth are the body's own as well as Gly-His-Lys-Cu substances marketed synthetic product (EP-A-0 765 152), having, however, very low enzymatic stability. Also included herein is the hexapeptide Arg-Pro-Leu-Lys-Pro-Trp (WO-2005/082395) currently not ready for a market launch.

Newer research on promoting hair growth has been published in Biol. Pharm. Bull., 28(3), 485 -489, (2005) and in WO-2005/095441. Described therein is the water-soluble pentapeptide Gly-Pro-Ile-Gly-Ser that is beneficial for the growth of hair bulb keratinocytes, whereby the new growth of hair is stimulated in telogenic mice. However, this peptide does not meet the prerequisites for sufficient skin permeation.

The object of the invention is, therefore, to provide cosmetic active substances that promote hair growth demonstrating sufficient potency and have minimal physiologically disadvantageous side effects, if any, and that provide sufficient skin permeation as well as biological stability.

A first aspect of the invention achieves the object with compounds of the formula

R¹-Gly-Pro-Ile-Gly-A₁ (I),

where
A₁ stands for Ser or Thr and
R¹ denotes —C(═O)—H, —C(═O)—C₁-C₂₄ alkyl, —C(═O)—CH₂CH(OH)CH₂N⁺(CH₃)₃ (carnitinoyl), 1,2-dithiolane-3-pentanoyl (liponoyl), —C(═O)-(CH₂)₂NHC(═O)CH(OH)C(CH₃)₂CH₂OH (pantothenoyl)
or

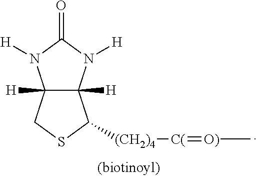

Compounds I can be present in the form of racemates or in enantiopure form as well as in free form or in salt form, preferably as a dermatologically tolerated salt, for example as a salt with a base such as hydroxide of sodium, potassium, calcium or ammonium, or as inner salts (zwitterions; betaine).

The substitution of the basic pentapeptide with a representative of the lipophilic group R¹ to form compounds I raises not only the log P value (measure of lipophilia/skin permeability) but, surprisingly, also considerably the stability of compounds I against enzymatic decomposition by peptidases, thus resulting in longer lasting stimulation of hair growth in comparison with the corresponding values for the unsubstituted basic peptide. Surprisingly, stronger hair bulb keratinocyte growth was documented as well.

“C₁-C₂₄ alkyl” is understood to mean straight-chained or branched, saturated hydrocarbon moieties having 1 to and including 24 carbon atoms, such as, for example, methyl, ethyl, n-propyl, n-butyl, n-pentyl, n-hexyl, n-heptyl, n-octyl, n-nonyl, n-undecyl, n-dodecyl, n-tridecyl, n-hexadecyl, n-heptadecyl, n-octadecyl, n-nonadecyl, i-propyl, tert.-butyl, i-butyl, sec-butyl or i-pentyl.

The three-letter abbreviations for amino acid components used previously and below have the following meanings: Ser=serine, Thr=threonine, Gly=glycine, Pro=proline, Ile=isoleucine.

In a preferred embodied example of the invention, the compounds I are enantiopure, starting from natural amino acids. In another preferred embodied example A₁ is Ser in the compounds I. Preferred specific compounds I are listed in Table 1.

| No. | R¹ | A₁ | Remarks |
|---|---|---|---|
| 1 | —C(═O)—H | Ser | |
| 2 | —C(═O)—CH₃ | Ser | |
| 3 | —C(═O)—C(CH₃)₃ | Ser | |
| 4 | —C(═O)—(CH₂)₄CH₃ | Ser | |
| 5 | —C(═O)—(CH₂)₈CH₃ | Ser | |
| 6 | —C(═O)—(CH₂)₁₄CH₃ | Ser | |
| 7 | —C(═O)—CH₂CH(OH)CH₂N⁺(CH₃)₃ | Ser | As inner salt; |
| | | | from L-carnitine |
| 8 | —C(═O)—(CH₂)₂NHC(═O)CH(OH)C(CH₃)₂CH₂OH | Ser | From (R)-N-(2,4- |
| | | | dihydroxy-3,3- |
| | | | dimethyl-1-oxo- |
| | | | butyl)-beta-alanine |
| 9 | 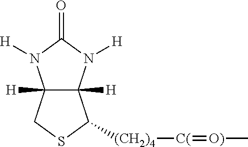 | Ser | From (3aS,4S,6aR)- biotine; mass spectrum: 654.50 = (M − H) |
| 10 | 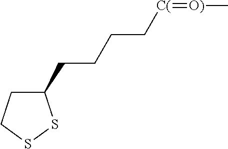 | Ser | From (R)-(+)-5- (1,2-dithiolan- 3-yl)-pentanoic acid |
| 11 | —C(═O)—H | Thr | |
| 12 | —C(═O)—CH₃ | Thr | |
| 13 | —C(═O)—C(CH₃)₃ | Thr | |
| 14 | —C(═O)—(CH₂)₄CH₃ | Thr | |
| 15 | —C(═O)—(CH₂)₈CH₃ | Thr | |
| 16 | —C(═O)—(CH₂)₁₄CH₃ | Thr | |
| 17 | —C(═O)—CH₂CH(OH)CH₂N⁺(CH₃)₃ | Thr | As inner salt; |
| | | | from L-carnitine |
| 18 | —C(═O)—(CH₂)₂NHC(═O)CH(OH)C(CH₃)₂CH₂OH | Thr | From (R)-N-(2,4- |
| | | | dihydroxy-3,3- |
| | | | dimethyl-1-oxo- |
| | | | butyl)-beta- |
| | | | alanine |
| 19 | 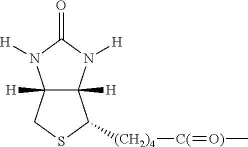 | Thr | From (3aS,4S,6aR)- biotine |
| 20 | 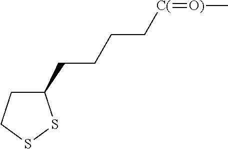 | Thr | From (R)-(+)-5- (1,2-dithiolan- 3-yl)-pentanoic acid |

In a special embodied example of the invention, the compounds I are selected from the group of compounds in free form or in salt form, (i) palmitoyl-Gly-Pro-Ile-Gly-Ser-OH; (ii) carnitinoyl-Gly-Pro-Ile-Gly-Ser-OH; (iii) 1,2-Di-thiolane-3-pentanoyl-Gly-Pro-Ile-Gly-Ser-OH; (iv) pantothenoyl-Gly-Pro-Ile-Gly-Ser-OH; and (v) 5-[(3aS,4S,6aR)-2-oxo-hexahydro-1H-thieno[3,4-d]imidazol-4-yl]pentanoyl-Gly-Pro-Ile-Gly-Ser-OH, preferably from those of this compound having a -Ser-O— partial structure such as, for example, carnitinoyl-Gly-Pro-Ile-Gly-Ser-O.

Compounds I can be prepared in accordance with known methods (for example, according to the general instructions in M. Bodanszky, “The Practice of Peptide Synthesis”, Springer, 2^{nd} Edition, 1994); for example, an amino acid A₁ having protected hydroxy amino and alpha amino groups, is coupled by means of the carboxyl function to a correspondingly functionalized solid phase; the alpha amino protective group (for example, a tert.-butyloxycarbonyl group) is then cleaved, now the peptide is build up step-by-step with the reagents that are commonly used in peptide synthesis until the desired pentapeptide has been completely synthesized; the released amino group is then transferred in position P1 of the peptide into a R¹—N(H) group, the thus obtained compound is then decoupled from the solid phase, the hydroxy protective group in position P5 of the peptide (for example, a benzyloxycarbonyl or tert.-butyloxycarbonyl group) is then cleaved and the obtained compound I is cleaned via chromatography and/or recrystallization.

The compounds I are preferably used as an active substance in cosmetic dermatologically active compositions, preferably at a concentration between 0.5 and 50000, particularly between 0.5 and 5000, especially between 5 and 50000, preferred between 1 and 500, preferably between 50 and 500, ppm (w/w).

A further aspect of the invention provides for the use of at least one of the compounds I as an active substance in the production of cosmetic dermatologically active, preferably topically applied, compositions, preferable for the promotion of hair growth.

A further aspect of the invention is directed to cosmetic dermatologically active compositions containing preferably between 0.5 and 50000, particularly between 0.5 and 5000, especially between 5 and 50000, preferredy between 1 and 500, preferably between 50 and 500, ppm (w/w) of at least one of the compounds I as active substance.

In compositions according to the invention, the compounds I can be used preferably in form of a solution, dispersion or emulsion, encapsulated in carrier substances, preferably in macro-, micro- or nano-capsules, liposomes or chylomicrons, enclosed in macro-, micro- or nano-particles or micro-sponges or absorbed on organic polymers in powder form or mineral carriers, preferably talcum or bentonite.

The compounds I can be used in the compositions in any dermatologically expedient galenic form, preferably in form of creams, lotions, ointments, shampoos, gels, powders, sprays, milk products, patches or body oils, preferably in form or water-in-oil or oil-in-water emulsions or gelatinizing and viscous, tensioactive and emulsifying polymers.

The compositions according to the invention are preferably used as cosmetically active, preferably topically applied agents, preferred as a hair growth promoter.

In a preferred embodied example, the composition according to the invention contains at least one additional cosmetic active (hair) care agent such as, for example, an extraction lipid, a synthesis lipid, a gelatinizing and viscous, tensioactive and emulsifying polymer, a principle of action soluble in water or oil, a plant extract, a tissue extract, a seawater extract, a sun screen agent, an antioxidant, a moisturizer, a barrier agent or a skin-revitalizing agent; especially preferred is at least one additional skin care agent selected from the group of hair growth promoters, preferably biotine, carnitine, pantothenoic acid, caffeine, minoxidil, carnosine, taurine, the ingredients of the Sophora root, procyanidins, vitamin A, vitamin D and arginine.

The compositions according to the invention preferably also contain a dermatologically acceptable carrier, meaning a carrier that is suitable for the dermatological, preferably topical, application on the skin, preferably with good esthetic properties, that is compatible with compounds I and preferably the other components as well and does not suffer from any safety- and toxicity-related disadvantages.

A carrier of this kind can be present in many different form. For example, emulsion carriers are suitable, including oil-in-water emulsions, water-in-oil emulsions, water-in-oil-in-water emulsions and oil-in-water-in-silicone emulsions; for example

A) Water-in-silicone emulsions containing a continuous silicone phase and an aqueous phase that is dispersed therein; or
B) Oil-in-water emulsions having a continuous aqueous phase and a hydrophobic phase that is dispersed therein and not soluble in water (“oil phase”). Examples of expedient oil-in-water emulsion carriers are specified in U.S. Pat. No. 5,073,371 and U.S. Pat. No. 5,073,372.

The respective quantities of cosmetic and/or dermatological excipients and auxiliary material that must be used can be easily ascertained by a person skilled in the art applying a trial and error method.

A further aspect of the invention is directed at a cosmetic method for promoting hair growth providing that at least one compound or composition according to the invention is to be applied on the hair, preferably the scalp area; wherein this application occurs once or multiple times such as, for example, once daily, more than once daily, once every other day or in other suitable intervals over the course of a certain time period or permanently.

The invention will be illustrated in further detail using specific examples below. These embodied example are not intended to limit the scope of protection of the invention (“wt-%” stands for weight percent).

### EXAMPLE 1

### Hair Foam and Shower Agent

A hair foam and shower agent is prepared analogous to the known methods according to the following formulation:

| Phase | Ingredients | INCI Name | Wt-% |
|---|---|---|---|
| A | Distilled water | Aqua | 26.00 |
| | Carbopol AQUA SF-1 | Acrylates Copolymer | 7.50 |
| | polymer | | |
| | Texapon NSO-BZ | Sodium Laureth Sulfate | 40.00 |
| B | Miranol Ultra C 32 | Sodium Cocoamphoacetate | 5.00 |
| | Hostapon CLG | Sodium Lauroyl Glutamate | 4.50 |
| C | JaguarC162 | Hydroxypropyl Guar Hydroxypropyltrimonium | 10.00 |
| | (Premix 2%) | Chloride | |
| | Pentapeptide derivative | — | 0.0025 |
| | no. 9 from Table 1 | | |
| | Distilled water | Aqua | 0.7475 |
| | Glycerin | Glycerin | 1.75 |
| | Euxyl K 300 | Phenoxyethanol (and) Methylparaben (and) | 0.80 |
| | | Ethylparaben (and) Propylparaben (and) | |
| | | Butylparaben (and) Isobutylparaben | |
| | Lime parfume | Fragrance | 0.50 |
| | FD & C Yellow No. 5 | Cl 19140 | q. s. |
| | Frescolat Plus | Menthyl Lactate (and) Menthol | 0.20 |
| D | Dehyton AB-30 | Coco-Betaine | 2.00 |
| | Rewoderm LI S 80 | PEG-200 Hydrogenated Glyceryl Palmate | 1.00 |
| | | (and) PEG-7 Glyceryl Cocoate | |
| E | Citric acid | Citric Acid | q. s. |

### EXAMPLE 2

### Hair and Scalp Agent

A hair and scalp agent is prepared analogous to the known methods according to the following formulation:

| Phase | Ingredients | INCI Name | Wt-% |
|---|---|---|---|
| A | Distilled water | Aqua | 63.3875 |
| | Ethanol DEB 96% | Alcohol denat. | 30.00 |
| | PVP/VA Copolymer | PVP/VA Copolymer | 2.50 |
| | Euxyl K 300 | Phenoxyethanol (and) Methylparaben (and) | 0.80 |
| | | Ethylparaben (and) Propylparaben (and) | |
| | | Butylparaben (and) Isobutylparaben | |
| B | PROTACHEM HCO-40 | PEG-40 Hydrogenated Castor Oil | 0.50 |
| | Parfume | Fragrance | 0.10 |
| C | Triethanolamine 99% | Triethanolamine | 0.01 |
| D | FD & C Yellow No. 5 | Cl 19140, Aqua | 0.10 |
| | (solution 0.5%) | | |
| | FD&C Blue No. 1 | Cl 42090, Aqua | 0.10 |
| | (solution 0.5%) | | |
| | Pentapeptide derivative | — | 0.0025 |
| | No. 9 from table 1 | | |
| | Distilled water | Aqua | 0.75 |
| | Glycerin | Glycerin | 1.75 |

### EXAMPLE 3

### Lotion

A lotion is prepared analogous to the known methods according to the following formulation:

| Phase | Ingredients | INCI Name | Wt-% |
|---|---|---|---|
| A | Pationic138C | Sodium Lauroyl Lactylate | 0.34 |
| | Cetyl Alcohol | Cetyl Alcohol | 2.00 |
| | Tegin 4100 | Glyceryl Stearate | 2.00 |
| | Tegosoft TN | C12-15 Alkyl Benzoate | 7.00 |
| | Tegosoft CT | Caprylic/Capric Triglycerides | 7.00 |
| B | Distilled water | Aqua | 76.455 |
| | Propylene glycol | Propylene Glycol | 5.00 |
| | Pentapeptide derivative | — | 0.005 |
| | no. 9 from table 1 | | |
| | Keltrol RD | Xanthan Gum | 0.20 |
| C | Acid or base for adjusting | — | q. s. |
| | the pH to a value | | |
| | between 5.0 to 5.5 | | |

Phases A and B are heated separately to 75° C. Phase A is then added to phase B while stirring, the mixture is homogenized, then allowed to cool to room temperature. Afterwards the pH is adjusted to 5.0 to 5.5 using phase 3.

### EXAMPLE 4

### Cream

A cream is prepared analogous to the known methods according to the following formulation:

| Phase | Ingredients | INCI Name | Wt-% |
|---|---|---|---|
| A | Imwitor 372P | Glyceryl Stearate Ditrate | 2.00 |
| | Cutina GMS | Glyceryl Stearate | 3.00 |
| | Sympatens-O/4200 | Sorbitan Laurate (and) Polyglyceryl-10 | 1.00 |
| | | Laurate | |
| | Almond oil | Prunus Amygdalus Dulcis | 2.50 |
| | Tegosoft TN | C12-15 Alkyl Benzoate | 7.00 |
| | Cetiol OE | Dicaprylyl Ether | 5.00 |
| | Tegosoft DC | Decyl Cocoate | 3.00 |
| | Butylhydroxytoluol | BHT | 0.05 |
| | Euxyl PE9010 | Phenoxyethanol (and) Ethylhexylglycerin | 1.00 |
| | Dow Corning 345 | Cyclopentasiloxane (and) Cyclohexasiloxane | 2.00 |
| B | Keltrol RD | Xanthan Gum | 0.30 |
| | Glycerin | Glycerin | 2.00 |
| | Distilled water | Aqua | 68.345 |
| C | Pentapeptide derivative | — | 0.005 |
| | no. 9 from table 1 | | |
| | Distilled water | Aqua | 0.75 |
| | Glycerin | Glycerin | 1.75 |
| D | Rapithix A-60 | Sodium Polyacrylate (and) Hydrogenated | 0.30 |
| | | Polydecene (and) Trideceth-6 | |
| E | Acid or base for adjusting | | q. s. |
| | the pH to a value between | | |
| | 5.0 to 5.5 | | |

### EXAMPLE 5

### Detection of the Hair Bulb Keratinocyte Proliferation

In the test as described in Biol. Pharm. Bull., 28(3), 485 - 489, (2005), compounds I demonstrate a more pronounced keratinocyte growth in comparison to Gly-Pro-Ile-Gly-Ser. For example, the test result for pentapeptide derivate no. 9 from table 1 is a value that is 53% superior in comparison with the corresponding value for Gly-Pro-Ile-Gly-Ser.

1. A compound of formula
R¹-Gly-Pro-Ile-Gly-A₁ (I),
where
A₁ stands for Ser or Thr and
R¹ denotes —C(═O)—H, —C(═O)—C₁-C₂₄ alkyl, —C(═O)—CH₂CH(OH)CH₂N⁺(CH₃)₃ (carnitinoyl), 1,2-Dithiolane-3-pentanoyl (liponoyl), —C(═O)-(CH₂)₂NHC(═O)CH(OH)C(CH₃)₂CH₂OH (pantothenoyl) or 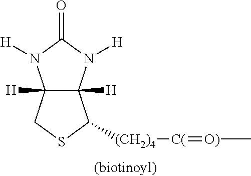
in a free form or a salt form.

2. The compound according to claim 1 being in racemate or enantiopure form.

3. The compound according to claim 1 wherein A₁ stands for Ser.

4. The compound according to claim 1, being selected from the group of the compounds, in free form or in salt form, (i) palmitoyl-Gly-Pro-lle-Gly-Ser-OH; (ii) carnitinoyl-Gly-Pro-lle-Gly-Ser-OH; (iii) 1,2-dithiolane-3-pentanoyl-Gly-Pro-Ile-Gly-Ser-OH; (iv) pantothenoyl-Gly-Pro-Ile-Gly-Ser-OH; and (v) 5-[(3aS,4S,6aR)-2-oxo-hexahydro-1H-thieno[3,4-d]imidazol-4-yl]pentanoyl-Gly-Pro-Ile-Gly-Ser-OH.

5. The compound according to claim 1 comprising carnitinoyl-Gly-Pro-Ile-Gly-Ser-O.

6. (canceled)

7. A cosmetic dermatologically active composition comprising at least one compound as defined in formula I of claim 1 as an active substance.

8. The composition according to claim 7, containing between 0.5 and 50000 ppm (w/w) active substance.

9. The composition according to claim 7, containing at least one further skin care agent selected from the group of hair growth promoter.

10. The composition according to claim 7, wherein the active substance being in a form of a solution, a dispersion or an emulsion.

11. The composition according to claim 7 being in a form of a cream, a lotion, an ointment, a shampoo, a gel, a powder, a spray, a milk product, a patch or a body oil.

12. (canceled)

13. A cosmetic method for promoting hair growth, comprising the step of: providing a composition of claim 1.

14. The compound of claim 1 wherein the salt being a dermatologically tolerated salt.

15. The composition of claim 7 containing between 0.5 and 5000 ppm (w/w) active substance.

16. The composition of claim 7 containing between 5 and 50000 ppm (w/w) active substance.

17. The composition of claim 7 containing between 1 and 500 ppm (w/w) active substance.

18. The composition of claim 7 containing between 50 and 500 ppm (w/w) active substance.

19. The composition of claim 9 wherein the hair growth promoters being selected from the group consisting of: biotine, carnitine, pantothenoic acid, caffeine, minoxidil, carnosine, taurine, the ingredients of the Sophora root, procyanidins, vitamin A, vitamin D and arginine.

20. The composition of claim 7 wherein the active substance being encapsulated in a carrier substance.

21. The composition of claim 20 wherein the carrier substances being selected from the group consisting of: macro-, micro- or nano-capsules, liposomes or chylomicrons, enclosed in macro-, micro- or nano-particles or micro-sponges or absorbed on organic polymers in powder form or mineral carriers.

22. The composition of claim 7 being in a form of a water-in-oil or oil-in-water emulsion or a gelatinizing and viscous tensioactive and emulsifying polymer.
